(19) United States
(12) Patent Application Publication
(10) Pub. No.: US 2007/0066880 A1
(43) Pub. Date: Mar. 22, 2007

(54) Image-based probe guidance system

(76) Inventors: Warren Lee, Clifton Park, NY (US); Mir Said Seyed-Bolorforosh, Guilderland, NY (US); Douglas Glenn Wildes, Ballston Lake, NY (US); Lowell Scott Smith, Niskayuna, NY (US); Kai Erik Thomenius, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/223,354

(22) Filed: Sep. 9, 2005

(51) Int. Cl. A61B 5/05

(52) U.S. Cl. 600/407

(57) ABSTRACT

A system for guiding probe is presented. The system includes a probe configured to acquire image data representative of a region of interest. Additionally, the system includes an imaging system in operative association with the probe and configured to facilitate guiding the probe to a desirable location based on the acquired image data and indications of change in position of the probe.

### BACKGROUND

The invention relates generally to diagnostic imaging, and more particularly to the guidance of probes used for treatment or monitoring of regions of interest.

Heart rhythm problems or cardiac arrhythmias are a major cause of mortality and morbidity. Atrial fibrillation is one of the most common sustained cardiac arrhythmia encountered in clinical practice. Cardiac electrophysiology has evolved into a clinical tool to diagnose these cardiac arrhythmias. As will be appreciated, during electrophysiological studies, probes, such as multipolar catheters, are positioned inside the anatomy, such as the heart, and electrical recordings are made from the different chambers of the heart.

Conventional catheter-based techniques used in interventional procedures typically involve inserting a probe, such as an imaging catheter, into a vein, such as the femoral vein. Prior to performing catheter-based interventional procedures, where an imaging catheter is used for either monitoring or treatment, precise guidance of the imaging catheter from the point of entry, through the vasculature of the patient to the desirable anatomical location is progressively becoming more important. Current techniques typically employ fluoroscopy to monitor and guide the imaging catheter within the vasculature.

A drawback of these techniques however is that these procedures are extremely tedious requiring considerable manpower, time and expense. Further, the long procedure times associated with the currently available catheter-based interventional techniques increase the risks associated with long term exposure to ionizing radiation to the patient as well as medical personnel. Additionally, fluoroscopy disadvantageously suffers from drawbacks, such as difficulty in visualizing soft tissues.

Additionally, probe-based imaging techniques may also be employed in industrial applications, such as inspection of regions within industrial parts. For example, currently available techniques employ a probe-based system to inspect piping in industrial parts or pipelines, turbine blades, and liquid reactors. However, these techniques are extremely tedious requiring considerable manpower, time and expense. Further, current techniques disadvantageously suffer from drawbacks such as difficulty in inspecting parts such as turbine blades with complex internal cooling structures and liquid reactors including enclosed structures like heat exchangers and/or containment vessels.

There is therefore a need for an image-based probe guidance system for monitoring and/or treating regions of interest. In particular, there is a significant need for a design that advantageously enhances probe placement and guidance thereby eliminating the need for harmful exposure to ionizing radiation.

### BRIEF DESCRIPTION

In accordance with aspects of the present technique, a system for guiding a probe is presented. The system includes a probe configured to acquire image data representative of a region of interest. Additionally, the system includes an imaging system in operative association with the probe and configured to facilitate guiding the probe to a desirable location based on the acquired image data and indications of change in position of the probe.

In accordance with another aspect of the present technique, a method for guiding a probe is presented. The method includes providing a probe configured to acquire image data representative of a region of interest. Further, the method includes providing an imaging system in operative association with the probe and configured to facilitate guiding the probe to a desirable location based on the acquired image data and indications of change in position of the probe.

In accordance with further aspects of the present technique a method for guiding a probe is presented. The method includes acquiring image data via a probe. In addition, the method includes monitoring a change in position of the probe. The method also includes providing updated information regarding probe position to facilitate guiding the probe to a desirable location. Computer-readable medium that afford functionality of the type defined by this method is also contemplated in conjunction with the present technique.

In accordance with further aspects of the present technique a system for guiding a probe is presented. The system includes a probe configured to image a region of interest. Additionally, the system includes an imaging system in operative association with the probe and having a display area and a user interface area, where the imaging system is configured to receive acquired image data, generate an image of the region of interest based on the acquired image data, display the image on the display area of the imaging system, monitor a change in position of the probe, and provide updated information regarding probe position to facilitate guiding the probe to a desirable location.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an exemplary image-based probe guidance system, in accordance with aspects of the present technique;

FIG. 2 is a front view of a display area of the image-based probe guidance system of FIG. 1, in accordance with aspects of the present technique;

FIG. 3 is a flow chart illustrating an exemplary process of guiding a probe, in accordance with aspects of the present technique;

FIGS. 4-5 illustrate the functioning of the exemplary image-based probe guidance system illustrated in FIG. 1, in accordance with aspects of the present technique; and

FIGS. 6-10 illustrate the functioning of the exemplary image-based probe guidance system and updating of associated historical records, in accordance with aspects of the present technique.

### DETAILED DESCRIPTION

As will be described in detail hereinafter, an automated image-guided system and method in accordance with exemplary aspects of the present technique are presented. Prior to performing a catheter-based interventional procedure where an imaging catheter is employed for either monitoring or treatment, it is desirable to guide the imaging catheter from the point of entry through the vascular system of a patient and to the desirable destination within the vasculature of the patient. Based on image data acquired by an imaging system via an imaging catheter, a user may guide the imaging catheter to a desirable anatomical location within the patient.

Although, the exemplary embodiments illustrated hereinafter are described in the context of a medical imaging system, it will be appreciated that use of the image-based probe guidance system in industrial applications are also contemplated in conjunction with the present technique.

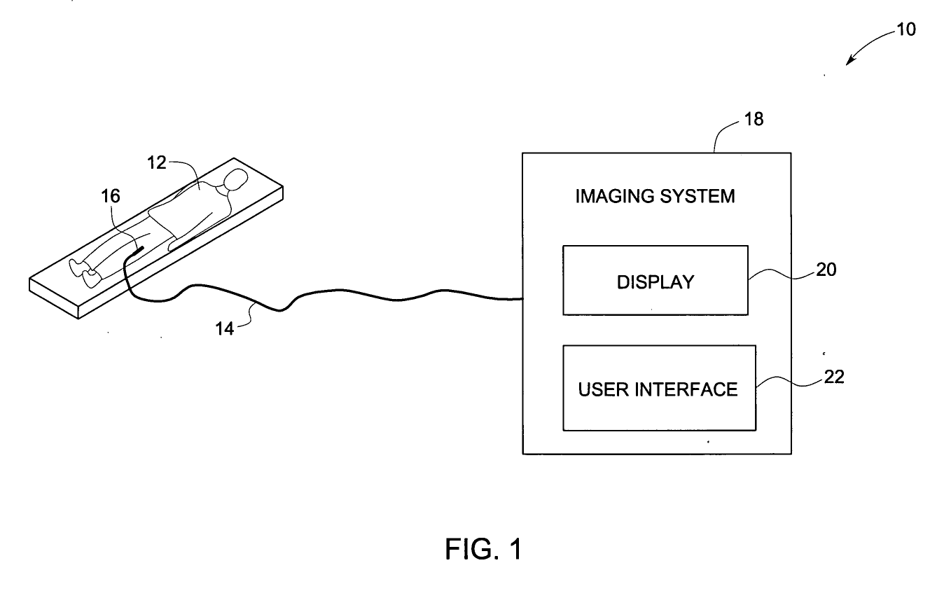

FIG. 1 is a block diagram of an exemplary system 10 for use in guiding a probe in accordance with aspects of the present technique. The image-based probe guidance system 10 may be configured to facilitate acquisition of image data from a patient 12 via a probe 14. In other words, the probe 14 may be configured to acquire image data representative of a region of interest in the patient 12, for example. In accordance with aspects of the present technique, the probe 14 may be configured to facilitate interventional procedures. It should also be noted that although the embodiments illustrated are described in the context of a catheter-based probe, other types of probes such as endoscopes, laparascopes, surgical probes, probes adapted for interventional procedures, or combinations thereof are also contemplated in conjunction with the present technique. An external probe may also be employed in situations where a user such as a sonographer guiding an imaging procedure is located at a remote location and therefore unable to see the probe or patient. Reference numeral 16 is representative of a portion of the probe 14 disposed inside the vasculature of the patient 12.

In certain embodiments, the probe may include an imaging catheter-based probe 14. Further, an imaging orientation of the imaging catheter 14 may include a forward viewing catheter or a side viewing catheter. However, a combination of forward viewing and side viewing catheters may also be employed as the imaging catheter 14. The imaging catheter 14 may include a real-time imaging transducer (not shown).

As previously noted, the imaging catheter 14 may be configured to facilitate acquisition of image data from the patient 12. Additionally, in accordance with aspects of the present technique, the imaging catheter 14 may be configured to facilitate guidance of the imaging catheter 14 within the vasculature of the patient 12 based on the acquired image data. The process of guiding the probe 14 within the vasculature of the patient 12 based on the image data acquired via the imaging catheter 14 will be described in greater detail hereinafter.

The system 10 may also include an imaging system 18 that is in operative association with the imaging catheter 14 and configured to facilitate guiding the imaging catheter 14 to a desirable location. In accordance with exemplary aspects of the present technique, the imaging system 18 may be configured to guide the imaging catheter 14 to a desirable location based on the acquired image data and indications of change in position of the imaging catheter 14. It should be noted that although the exemplary embodiments illustrated hereinafter are described in the context of a medical imaging system, such as an ultrasound system, other imaging systems such as, but not limited to, optical imaging systems, pipeline inspection systems, liquid reactor inspection systems, or other imaging systems are also contemplated for guiding the imaging catheter 14 to a desirable location.

In accordance with aspects of the present technique, the imaging system 18 may be configured to generate a current image based on the acquired image data. As used herein, “current” image embodies an image representative of the current position of the imaging catheter 14. Accordingly the imaging system 18 may be configured to acquire image data representative of an anatomical region of the patient 12 via the imaging catheter 14. While image data may be directly acquired from the patient 12 via the imaging catheter 14, the imaging system 18 may instead acquire stored image data representative of the anatomical region of the patient 12 from an archive site or data storage facility.

Further, the imaging system 18 may be configured to display the generated image representative of a current position of the imaging catheter 14 within a region of interest in the patient 12. As illustrated in FIG. 1, the imaging system 18 may include a display area 20 and a user interface area 22. In accordance with aspects of the present technique, the display area 20 of the imaging system 18 may be configured to display the image generated by the imaging system 18 based on the image data acquired via the imaging catheter 14. Additionally, the display area 20 may be configured to aid the user in visualizing the generated image.

Moreover, the imaging system 18 may also be configured to detect a change in position of the imaging catheter 14 by monitoring the position of the imaging catheter 14 within the vasculature of the patient 12. Accordingly, the progression of the imaging catheter 14 within the vasculature of the patient 12 may be visualized by displaying a “history image” on a portion of the display area 20 of the imaging system 18. As used herein, the “history” image is representative of the progress of the imaging catheter 14 within the vasculature of the patient 12. Accordingly, the imaging system 18 may be configured to facilitate monitoring of the position of the imaging catheter 14 by comparing the acquired image data with predetermined information, thereby sensing a change in position of the imaging catheter 14. In certain embodiments, the predetermined information may include speckle targets.

It should be noted that the current image may include an image having a relatively higher resolution over a comparatively limited field of view. However, the history image may include a volumetric image having a comparatively lower resolution.

Further, the user interface area 22 of the imaging system 18 may include a human interface device (not shown) configured to facilitate the user to manipulate the guidance of the imaging catheter 14 within the vasculature of the patient 12. The human interface device may include a mouse-type device, a trackball, a joystick, or a stylus. However, as will be appreciated, other human interface devices, such as, but not limited to, a touch screen, may also be employed.

Additionally, a larger context to aid in the visualization and guidance of the imaging catheter 14 may be provided by coalescing the images generated based on image data acquired via the imaging catheter 14 with previously acquired images of the anatomical region being imaged. Accordingly, the imaging system 18 may also include a workstation (not shown) configured to register the generated images with previously acquired images of the region of interest being imaged. The previously acquired images may include images acquired via a variety of imaging techniques including, but not limited to, a computed tomography (CT) image, a magnetic resonance image (MR), an X-ray image, a nuclear medicine image, a positron emission tomography (PET) image, images acquired via other developing techniques, or combinations thereof. Additionally, the workstation may be configured to display the registered images on the display area 20 of the imaging system 18.

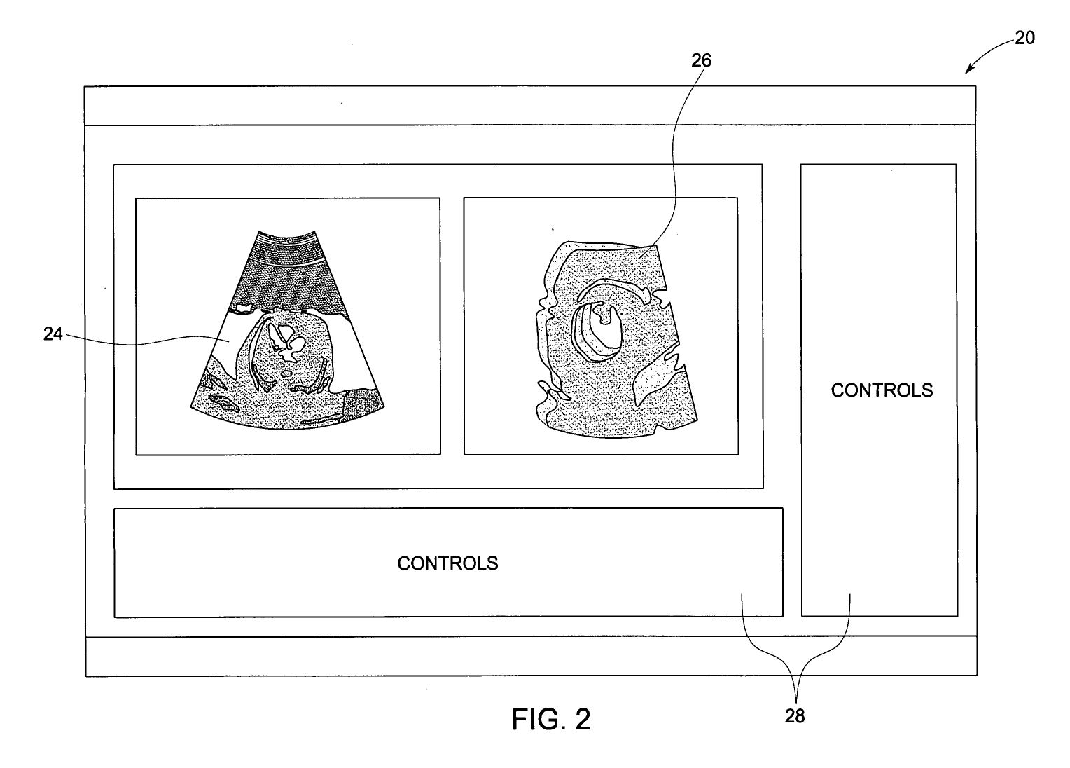

Turning now to FIG. 2, a front view of the display area 20 of the imaging system 18 of FIG. 1 is illustrated. As depicted in FIG. 2, the display area 20 may be configured to display a current image generated by the imaging system 18 (see FIG. 1) as well as the history image. As previously noted, the current image embodies an image representative of the current position of the imaging catheter 14. Reference numeral 24 is representative of the current image generated by the imaging system 18 based on the image data acquired via the imaging catheter 14 (see FIG. 1) from an anatomical region of the patient 12 (see FIG. 1). In one embodiment, the image 24 may correspond to a live image, where the live image is generated based on image data acquired in real-time. Further, reference numeral 26 embodies the history image generated by the imaging system 18, where the history image is representative of the progress of the imaging catheter 14 within the vasculature of the patient 12. In addition, the display area 20 may include controls 28 that may facilitate the user to manipulate the images displayed on the display area 20.

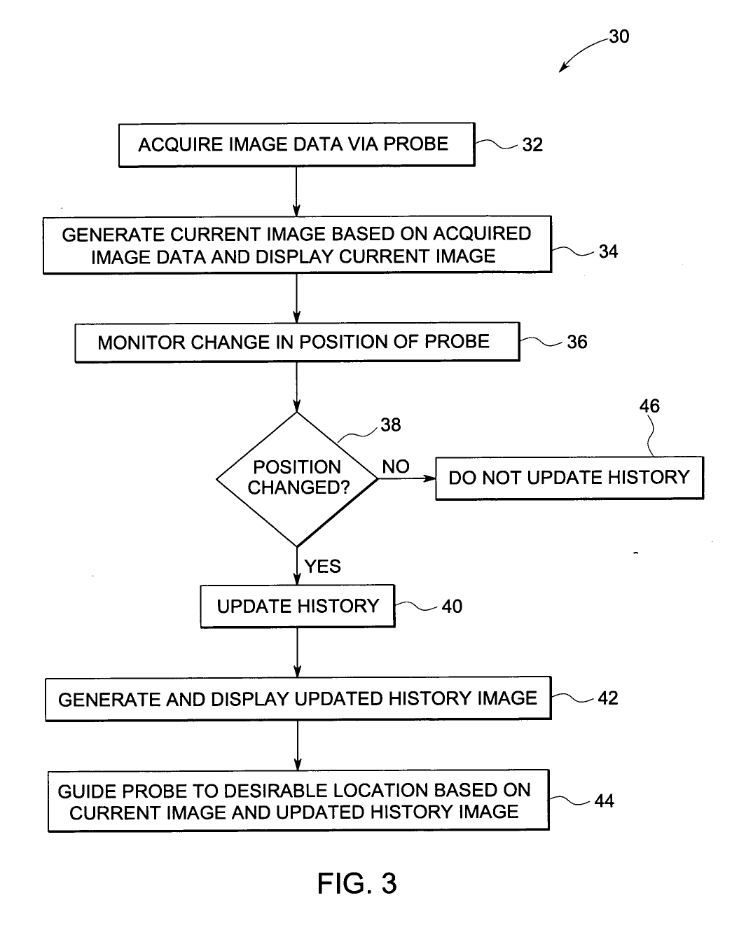

FIG. 3 is a flow chart of exemplary logic 30 for guiding a probe. In accordance with exemplary aspects of the present technique, a method for guiding the probe based on acquired image data is presented. The method starts at step 32 where image data representative of an anatomical region of the patient 12 (see FIG. 1) may be acquired by the imaging system 18 (see FIG. 1) via a probe, such as the imaging catheter 14 (see FIG. 1). The image data may be acquired in real-time employing the imaging catheter. This acquisition of image data via the imaging catheter aids a user in guiding the imaging catheter to a desirable location. It should be noted that mechanical means, electronic means, or combinations thereof may be employed to facilitate the acquisition of image data via the imaging catheter. Alternatively, previously stored image data representative of the anatomical region may be acquired by the imaging system 18. The imaging catheter may include an imaging transducer. Further, the imaging orientation of the imaging catheter may include a forward viewing catheter, a side viewing catheter or combinations thereof, as previously described.

Subsequently, at step 34, an image based on image data acquired by the imaging system 18 via the imaging catheter 14 is generated. This generated image may include a current image, as previously noted. Also, at step 34, the generated image representative of current data (“current image”) may be displayed on a portion of the display area 20 (see FIG. 2) to aid the user in visualizing the region of interest being imaged. Additionally, the generated image may be coalesced with previously acquired images of the region of interest being imaged. Accordingly, the generated image may be registered with the previously acquired images of the region of interest being imaged. Anatomical landmarks, such as, but not limited to, vessel branching, that are visible in both sets of images may be used to align the generated image and the previously acquired CT, MR, X-ray, PET or nuclear medicine images. It should be noted that the generated image, the previously acquired CT, MR and X-ray images or both may experience some stretching or distortion to compensate for motion of the patient body to obtain good image registration. Further, as previously noted, the registered images may be displayed on the display area 20.

The current image displayed on the display area 20 may then be processed at step 36 to monitor change in position of the imaging catheter 14. At step 36, the current image may be compared with predetermined information to detect change in position of the imaging catheter 14. The predetermined information may include speckle targets, for example. This comparison of the current image and the predetermined information may be achieved by processing the current image via image processing algorithms. The image processing algorithms may include, for example, correlation-based algorithms, speckle tracking algorithms, displacement sensing algorithms, imaging reconstruction algorithms, or combinations thereof.

Also, in one embodiment, the imaging catheter 14 may optionally include a position sensor disposed on a tip of the imaging catheter 14. The position sensor may be configured to track change in position of the imaging catheter 14 within the anatomy of the patient 12. Subsequently, the imaging system 18 may be configured to acquire the location information from the position sensor. In one embodiment, location information may be obtained from the position sensor by localization of the position sensor with respect to fixed points. For example, electromagnetic and/or optical ranging from fixed points, such as fixed sources, reflectors or transponders may be utilized to acquire the location information. Alternatively, in certain other embodiments, location information from the position sensor may be obtained via integration of velocity or acceleration changes from a known reference point. For example, mechanical gyroscopes or optical gyroscopes that respond to changes in velocity and/or acceleration may be employed to obtain the location information from the position sensor. Steps configured to facilitate detection of change in position of the imaging catheter 14 will be described in greater detail with reference to FIGS. 4-10.

Subsequently, at step 38, a check is carried out to verify if the imaging catheter 14 has been repositioned. If the imaging system 18 detects change in position of the imaging catheter 14 within the vasculature of the patient, the history image 26 (see FIG. 2) of the anatomical region being imaged may be updated at step 40. Following step 40, an updated history image may be generated and displayed on the display area 20 of the imaging system 18 at step 42. The steps of updating history 40 and generating and displaying updated history image 42 will be described in greater detail with reference to FIGS. 4-10.

Following step 42, the user may visualize both the current image and the updated history image displayed on the display area 20 of the imaging system 18. Subsequently, at step 44, updated information regarding the position of the imaging catheter may be provided to the imaging system 18. Consequently, the user may employ both the current image and the updated history image to guide the imaging catheter 14 to a desirable location. With returning reference to the decision block 38, if the imaging system 18 does not detect any change in position of the imaging catheter 14, then the history image is not updated as indicated by step 46.

Referring now to FIGS. 4-5 the functioning of the exemplary image-based probe guidance system 10 illustrated in FIG. 1 is exemplified. As will be appreciated by one skilled in the art, the figures are for illustrative purposes and are not drawn to scale. FIG. 4 illustrates a step 48 in the functioning of the image-based probe guidance system 10 (see FIG. 1). As shown in FIG. 4, an imaging catheter 50 currently disposed within the vasculature, such as a blood vessel 52 of the patient is depicted. Reference numerals 51, 53 and 55 embody an adventitia, a media and an intima of the blood vessel 52 respectively. In a presently contemplated configuration, the imaging catheter 50 may include a forward viewing catheter. However, as previously described, a side viewing catheter may also be employed.

Reference numeral 54 is representative of a field of view of the imaging catheter 50. It should be noted that in accordance with aspects of the present technique, the displayed current image may be analyzed in order to track change in position of the imaging catheter 50. In certain embodiments, a selected region of the displayed current image may be employed for the analysis. Alternatively, the whole displayed current image may be used to detect change in position of the imaging catheter 50. In the illustrated embodiment of FIG. 4, reference numeral 56 embodies a selected region of the displayed image between an upper bound 58 and a lower bound 60. Furthermore, reference numeral 62 corresponds to a distance traversed by the imaging catheter 50 within the vasculature 52.

As previously described, image processing algorithms, such as speckle tracking algorithms may be applied to the displayed current image to detect change in position of the imaging catheter 50. In one embodiment, the displayed current image may be processed via the application of a speckle tracking algorithm. As will be appreciated, speckle is directly related to ultrasonic scattering from tissue microstructure, and thus may be employed as a spatial marker for use in detecting change in position of the imaging catheter 50. By tracking image speckle from one frame to the next using correlation-based methods, very sensitive measures of tissue displacement may be produced. Accordingly, speckle targets 64 at an initial location within the selected region 56 of the displayed image may be identified.

Turning now to FIG. 5, a further step 66 in the functioning of the image-based probe guidance system 10 (see FIG. 1) is illustrated. As depicted in FIG. 5, the imaging catheter 50 has been advanced further into the vasculature 52. The new distance traversed by the imaging catheter 50 in the vasculature 52 is represented by reference numeral 68. It should be noted that the new distance 68 traversed by the imaging catheter 50 is greater than the distance 62 depicted in FIG. 4. Consequently, the location of the speckle targets 64 within the selected region 56 is relatively closer to the upper bound 58 as compared to the distance between the speckle targets 64 and the upper bound 58 of the selected region 56 of FIG. 4. The corresponding change in position of the imaging catheter 50 with respect to the speckle targets 64 may then be detected by the application of image processing algorithms. Subsequently, the change in position of the imaging catheter 50 detected by the imaging system causes the history image to be updated and displayed on the display area of the imaging system. For example, the history image may be updated by reconstructing the image to include new image information.

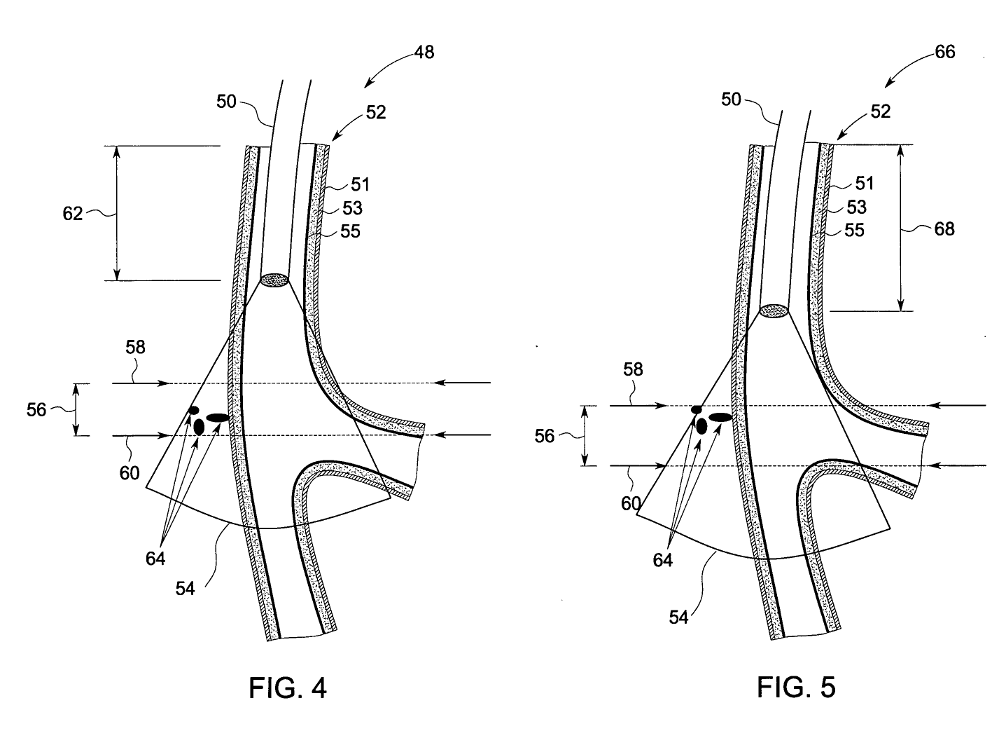

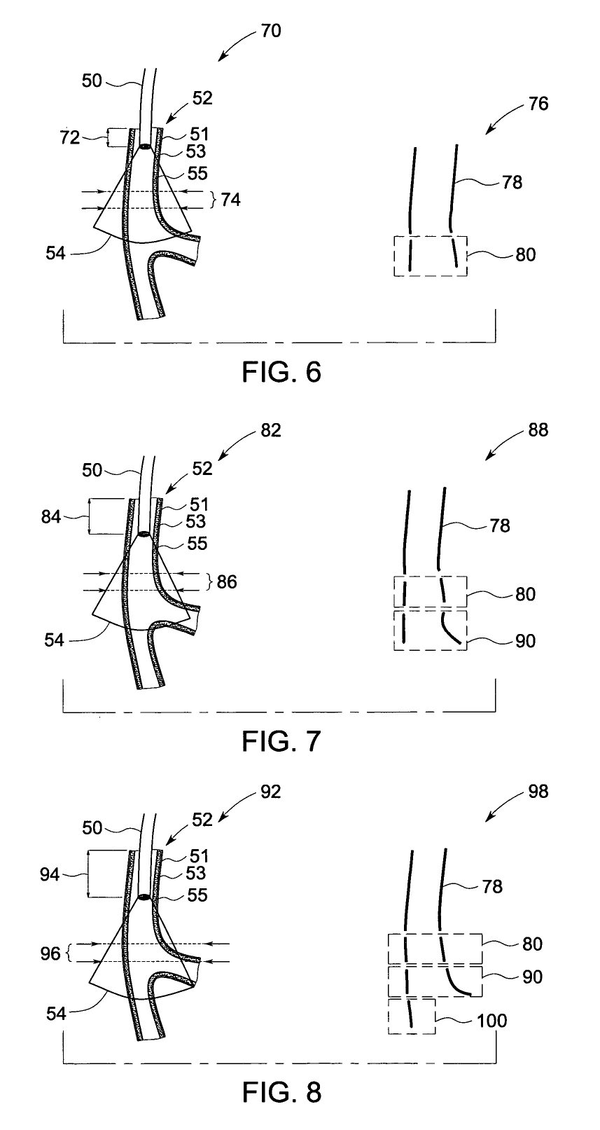

FIGS. 6-10 illustrate the functioning of the exemplary image-based probe guidance system and updating of associated historical images, in accordance with aspects of the present technique. As will be appreciated by one skilled in the art, the figures are for illustrative purposes and are not drawn to scale. In FIGS. 6-10, the change in position of the imaging catheter within the vasculature of a patient is illustrated. Also, a selected portion of each current image may be analyzed by the imaging system to monitor the progression of the imaging catheter as previously described with respect to FIGS. 4-5. It should also be noted that the selected portion of the current image may include a part of the current image or the whole current image. Further, it may be noted that with reference to FIGS. 6-10, reference numeral 50 represents an imaging catheter and reference numeral 52 embodies the vasculature. Also, as previously noted, reference numerals 51, 53 and 55 respectively embody an adventitia, a media and an intima of the blood vessel 52. Further, a field of view of the imaging catheter is represented by reference numeral 54, as previously described.

In the process step 70 illustrated in FIG. 6, reference numeral 72 signifies the distance traversed by the imaging catheter 50 within the vasculature 52. A selected portion of the current image is represented by reference numeral 74. The selected portion 74 of the image may then be analyzed to monitor change in position of the imaging catheter 50 as previously described with reference to FIGS. 4-5. Reference numeral 76 illustrates an embodiment of a history image. A history image 78 associated with a current position of the imaging catheter 50 is depicted. If a change in position of the imaging catheter 50 is detected, then the history image 78 is updated to include a portion 80 representative of the selected portion 74. Accordingly, the updated history image 76 is shown as including a portion 80 representative of an image associated with the selected region 74 and indicative of the change in position of the imaging catheter 50.

Further, FIG. 7 illustrates a process step 82 where the imaging catheter 50 has traversed a distance 84 within the vasculature 52, where the distance 84 is greater than the distance 72 depicted in FIG. 6. Image 88 depicts an updated history image of the history image 76 (see FIG. 6) including a portion 90 representative of an image associated with a selected region 86. Similarly, FIG. 8 depicts a process step 92. As indicated by the process step 92, the imaging catheter 50 has traversed a distance 94 within the vasculature 52. Further, image 98 represents an updated history image of history image 88 (see FIG. 7) including a portion 100 representative of an image associated with a selected region 96.

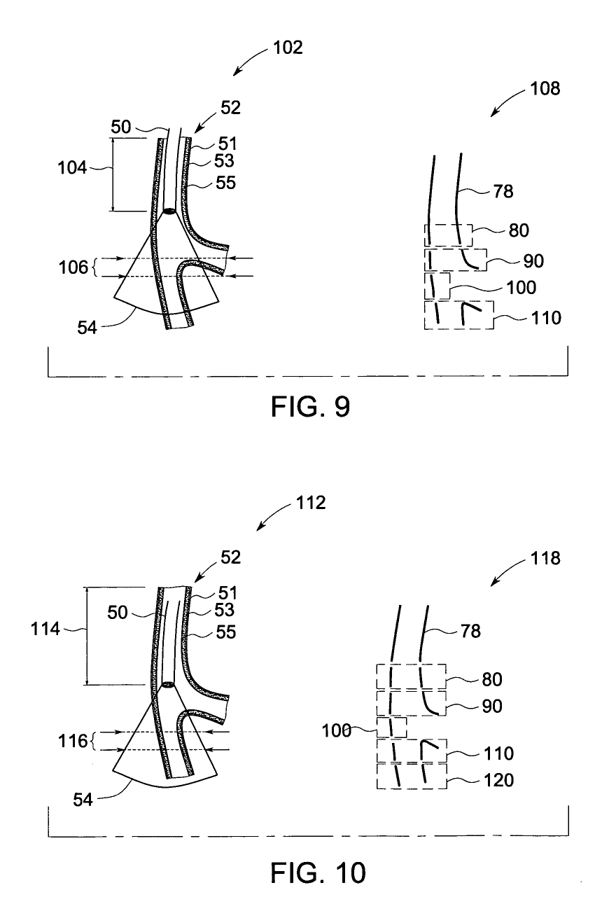

Likewise, FIG. 9 corresponds to a process step 102 where the imaging catheter 50 has traversed a distance 104 within the vasculature 52. Additionally, image 108 corresponds to an updated image of history image 98 (see FIG. 8) where image is updated to include a portion 110 corresponding to a selected region 106. In a similar fashion, FIG. 10 is representative of a process step 112. As indicated by the process step 112, the imaging catheter 50 has traversed a distance 114 within the vasculature 52. Further, image 118 represents an updated image of history image 108 (see FIG. 9) including a portion 120 representative of an image associated with a selected region 116.

As described with reference to FIGS. 6-10, the progression of the imaging catheter 50 within the vasculature 52 of the patient is tracked by analyzing a selected portion of each image generated based on acquired image data representative of a current location of the imaging catheter 50, where the selected portion may include a whole image or a part of the whole image. Based on the analysis, if change in position of the imaging catheter 50 is detected, the history image is updated. Consequently, while the imaging catheter 50 has only a limited field of view at a given instant, the reconstructed history image effectively widens the field of view and advantageously facilitates the user to determine the current position of the imaging catheter 50 and a direction in which the imaging catheter 50 needs to be guided to reach the desirable anatomical destination.

As will be appreciated by those of ordinary skill in the art, the foregoing example, demonstrations, and process steps may be implemented by suitable code on a processor-based system, such as a general-purpose or special-purpose computer. It should also be noted that different implementations of the present technique may perform some or all of the steps described herein in different orders or substantially concurrently, that is, in parallel. Furthermore, the functions may be implemented in a variety of programming languages, including but not limited to C++ or Java. Such code, as will be appreciated by those of ordinary skill in the art, may be stored or adapted for storage on one or more tangible, machine readable media, such as on memory chips, local or remote hard disks, optical disks (that is, CD's or DVD's), or other media, which may be accessed by a processor-based system to execute the stored code. Note that the tangible media may comprise paper or another suitable medium upon which the instructions are printed. For instance, the instructions can be electronically captured via optical scanning of the paper or other medium, then compiled, interpreted or otherwise processed in a suitable manner if necessary, and then stored in a computer memory.

The various methods of guiding a probe and the systems for guiding the probe described hereinabove dramatically enhance efficiency of the process of monitoring and guiding the probe through the vasculature of the patient without having to resort to using fluoroscopic methods currently employed to guide the probes, thereby advantageously eliminating exposure to harmful ionizing radiation required with current fluoroscopic imaging methods.

Also, the methods of guiding the probe described hereinabove use images generated based on image data acquired via the probe, thus greatly enhancing placement of the probe. Furthermore, a live image representative of the current location of the probe as well as the anatomical history of progression of the probe within the vasculature are simultaneously displayed on the display area in real-time, thereby advantageously facilitating the user to visualize the current location of the probe with respect to anatomical landmarks and subsequently guide the probe to a desirable anatomical destination. Additionally, employing the techniques of guiding the probe described hereinabove facilitates building cost effective probe guidance systems, as no additional hardware is required to implement the systems presented.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A system for guiding a probe, the system comprising:
a probe configured to acquire image data representative of a region of interest; and
an imaging system in operative association with the probe and configured to facilitate guiding the probe to a desirable location based on the acquired image data and indications of change in position of the probe.

2. The system of claim 1, wherein the probe is configured to facilitate interventional procedures.

3. The system of claim 1, wherein the probe comprises an imaging catheter, an endoscope, a laparascope, a surgical probe, or a probe adapted for interventional procedures.

4. The system of claim 3, wherein the imaging catheter comprises a forward viewing catheter, a side viewing catheter or combinations thereof.

5. The system of claim 1, wherein the imaging system is configured to:
acquire image data via the probe;
generate an image based on the acquired image data; and
display the generated image on a display area of the imaging system.

6. The system of claim 5, wherein the imaging system is further configured to sense change in position of the probe.

7. The system of claim 6, wherein the imaging system is configured to monitor position of the probe by updating a historical record of the region of interest being imaged, and displaying the historical record.

8. The system of claim 7, further configured to monitor the position of the probe by comparing the acquired image data with predetermined information to detect a change in position of the probe.

9. The system of claim 1, further comprising a workstation configured to register the generated images with previously acquired images of the region of interest being imaged and display the registered images on the display area.

10. The system of claim 9, wherein the previously acquired images comprise a computed tomography image, a magnetic resonance image, an X-ray image, a positron emission tomography image, a nuclear medicine image, or combinations thereof.

11. A method for guiding a probe, the method comprising:
providing a probe configured to acquire image data representative of a region of interest; and
providing an imaging system in operative association with the probe and configured to facilitate guiding the probe to a desirable location based on the acquired image data and indications of change in position of the probe.

12. The method of claim 11, further comprising:
acquiring image data via the probe;
generating an image based on the acquired image data; and
displaying the generated image on a display area of the imaging system.

13. The method of claim 12, further comprising sensing motion of the probe.

14. The method of claim 13, further comprising monitoring position of the probe by updating a historical record of the region of interest being imaged and displaying the historical record.

15. The method of claim 14, further comprising monitoring the position of the probe by comparing the acquired image data with predetermined information to detect a change in position of the probe.

16. The method of claim 12, further comprising registering the generated images with previously acquired images of the region of interest being imaged and displaying the generated images.

17. A method for guiding a probe, the method comprising:
acquiring image data via a probe;
monitoring a change in position of the probe; and
providing updated information regarding probe position to facilitate guiding the probe to a desirable location.

18. The method of claim 17, further comprising generating an image from the acquired image data for display on a display area of an imaging system.

19. The method of claim 18, further comprising registering the generated image with previously acquired images of the region of interest being imaged.

20. The method of claim 17, wherein the monitoring step comprises comparing the acquired image data with predetermined information to detect a change in position of the probe via applying image processing algorithms to the generated image.

21. The method of claim 20, wherein the image processing algorithms comprise correlation-based algorithms, speckle tracking algorithms, displacement sensing algorithms, image reconstruction algorithms, or combinations thereof.

22. A computer readable medium comprising one or more tangible media, wherein the one or more tangible media comprise:
code adapted to acquire image data via a probe;
code adapted to monitor a change in position of the probe; and
code adapted to provide updated information regarding probe position to facilitate guiding the probe to a desirable location.

23. The computer readable medium, as recited in claim 22, further comprising code adapted to generate an image from the acquired image data for display on a display area of an imaging system.

24. The computer readable medium, as recited in claim 23, further comprising code adapted to register the generated image with previously acquired images of the region of interest being imaged.

25. A system for guiding a probe, the system comprising:
a probe configured to image a region of interest;
an imaging system in operative association with the probe and having a display area and a user interface area, wherein the imaging system is configured to:
receive acquired image data;
generate an image of the region of interest based on the acquired image data;
display the image on the display area of the imaging system;
monitor a change in position of the probe; and
provide updated information regarding probe position to facilitate guiding the probe to a desirable location.

26. The system of claim 25, further comprising a workstation configured to register the image of the region of interest with previously acquired images of the region of interest, wherein the previously acquired images comprise a computed tomography image, a magnetic resonance image, an X-ray image, a positron emission tomography image, a nuclear medicine image, or combinations thereof.

27. The system of claim 25, further comprising an operator console configured to facilitate a user to guide the probe to the desirable location.

28. The system of claim 25, wherein the imaging system comprises an ultrasound imaging system, a magnetic resonance imaging system, an X-ray imaging system, a nuclear imaging system, a positron emission tomography system, or combinations thereof.

29. The system of claim 25, wherein the probe comprises an imaging catheter, an endoscope, a laparascope, a surgical probe, or a probe adapted for interventional procedures.
